(19) United States
(12) Patent Application Publication
(10) Pub. No.: US 2022/0096614 A1
(43) Pub. Date: Mar. 31, 2022

(54) PEPTIDE-INDUCED NK CELL ACTIVATION

(71) Applicant: University of Southampton, Southampton, Hampshire (GB)

(72) Inventor: Salim Iqbal Khakoo, Southampton (GB)

(73) Assignees: University of Southampton, Southampton, Hampshire (GB); University of Southampton, Southampton, Hampshire (GB)

(21) Appl. No.: 17/440,067

(22) PCT Filed: Mar. 23, 2020
(86) PCT No.: PCT/GB2020/050767

§ 371 Date: Sep. 16, 2021

(30) Foreign Application Priority Data
Mar. 21, 2019 (GB) 1903877.7

(51) Int. Cl. A61K 39/00; G01N 33/574; C12N 5/0783; A61P 35/00

(52) U.S. Cl. CPC A61K 39/0005; G01N 33/57492; C12N 5/0646; A61K 2039/605; A61K 2039/53; A61K 2039/585; A61P 35/00

(57) ABSTRACT

The invention relates to a method of treatment or prophylaxis of cancer, wherein the cancer overexpresses exportin-1, the method comprising the administration of: a peptide capable of activating NK cell-mediated immunity to cancer cells that overexpress exportin-1, the peptide comprising or consisting of the amino acid sequence XⁿAX²X¹, wherein Xⁿ is an amino acid sequence of between 5 and 12 residues, and X¹ is any amino acid; or leucine or phenylalanine; and X² is alanine, threonine or serine; or administration of one or more of a nucleic acid encoding the peptide; an immunogenic composition comprising the peptide; a complex comprising the peptide; a vesicle comprising the peptide or nucleic acid encoding the peptide; a dendritic cell comprising the peptide and/or comprising nucleic acid encoding the peptide; an activated NK cell, that has been activated by the peptide; or a virus or virus like particle comprising the peptide and/or comprising nucleic acid encoding the peptide.

This invention relates to NK cell activation and NK cell mediated immunity, immunogenic peptides, compositions and complexes; and associated methods of treatment or prophylaxis.

Natural killer (NK) cells are important in the immune response to cancer, inflammatory disorders and globally important infections, such as HIV, hepatitis B (HBV), hepatitis C (HCV), and malaria. Killer-cell immunoglobulin-like receptors (KIR) are expressed on NK cells and specific combinations of KIR and their HLA class I ligands lead to protection or susceptibility to HCV, HIV, HPV and malaria; to virus related cancers including hepatocellular carcinoma (HBV and HCV) and cervical cancer (HPV); to pregnancy associated disorders and the outcome of haematological malignancies and bone marrow transplantation. The KIR gene family has been implicated in head and neck cancer, colorectal cancer, lung cancer, leukemia, bladder cancer, gastric cancer, prostate cancer, breast cancer, neuroblastoma, glioblastoma, myeloma, lymphoma, and oral squamous cell carcinoma.

Virus-specific long-lived “memory” NK cells have been demonstrated in mice. Consistent with such populations in humans, expansions of NK cells expressing inhibitory or activating KIR specific for self-MHC class I have been observed in a number of viral infections (HCV, HIV, CMV, hantavirus) and associated with protection in both HCV and Chikungunya virus infection. The drivers for these self-MHC class I specific expansions are not clear. However, KIR engage MHC class I and its bound peptide and an unexpected sensitivity of NK cells to changes in the peptide content of MHC class I has been demonstrated.

The specificity of inhibitory receptors has been well defined. Due to the high sequence homology in the ligand binding domains with related inhibitory receptors, ligands for the activating KIR have been considered to be similar to those of the inhibitory KIR, but generally of lower affinity. Thus, in general activating KIR are thought to engage MHC class I molecules and its bound peptide with the same motifs as their inhibitory counterparts.

Previous studies described in patent publications WO2015170123 and WO2017081459 (both of which are herein incorporated by reference) have identified a series of peptides that can be used to activate NK cells, and provide NK cell-mediated killing for a broad range of diseases, such as viral infection and cancer.

An aim of the present invention is to provide an improved or alternative NK cell mediated therapy or prophylaxis.

According to a first aspect of the present invention, there is provided a peptide capable of activating NK cell-mediated immunity to cancer cells that overexpress exportin-1 (also known as crm1 and XPO-1), the peptide comprising or consisting of the amino acid sequence XⁿAX²X¹,
wherein
X^{a} is an amino acid sequence of between 5 and 12 residues, and
X¹ is any amino acid; or leucine or phenylalanine; and
X² is alanine, threonine or serine.

Advantageously, the invention herein has provided a previously unidentified therapeutic mechanism for treating cancers associated with overexpression of exportin-1. Nuclear export is mainly mediated by the nuclear export factor exportin-1 (XpoI, also known as chromosomal region maintenance 1, CRM1), and it is an essential function in all eukaryote that transport nuclear export signal (NES) containing cargoes from the nucleus to the cytoplasm. Upregulation of this process is a common characteristic for a broad spectrum of cancers, and it has been shown that inhibition of nuclear export kills cancer cells effectively (Sun et al. (2016. Signal Transduct Target Ther. 2016; 1: 16010), which is herein incorporated by reference). The invention advantageously identifies peptides that are capable of activating NK cell-mediate killing of cancer cells that overexpress exportin-1. Therefore, such peptides can be used in a vaccine approach for treatment of cancers associated with overexpression of exportin-1. The peptides of the invention beneficially and specifically target KIR2DS2+ NK cells, and KIR2DS2 recognises exportin-1 on tumours.

In one embodiment of the peptide X¹ is leucine or isoleucine. In one embodiment X¹ is leucine. Alternatively, in one embodiment X¹ is isoleucine. X¹ may be leucine. In another embodiment, X¹ is phenylalanine.

In one embodiment X² is alanine or threonine. In another embodiment X² is tryptophan or threonine. In another embodiment X² is alanine. In another embodiment X² is threonine. In another embodiment X² is tryptophan. In another embodiment, X² is serine.

In one embodiment X¹ is leucine and X² is alanine or threonine. In another embodiment X¹ is leucine and X² is tryptophan or threonine. In another embodiment X¹ is leucine and X² is tryptophan. In another embodiment X¹ is leucine and X² is threonine. In another embodiment X¹ is leucine and X² is alanine. In another embodiment, X¹ may be phenylalanine and X² may be threonine. In another embodiment, X¹ is leucine and X² is serine.

Xⁿ may be an amino acid sequence of 5 residues. Xⁿ may be an amino acid sequence of 6 residues. Xⁿ may be an amino acid sequence of 7 residues. Xⁿ may be an amino acid sequence of 5 residues and X¹ is leucine. Xⁿ may be an amino acid sequence of 6 residues and X¹ is leucine. Xⁿ may be an amino acid sequence of 7 residues and X¹ is leucine. Xⁿ may be an amino acid sequence of 7 residues and X² is tryptophan. Xⁿ may be an amino acid sequence of 7 residues and X² is threonine.

The peptide amino acid sequence may comprise any sequence selected from the group comprising:

LNPSVAATL; NPSVAATL; PSVAATL;
VAPWNAATL; APWNAATL; PWNAATL;
VAPWNSATL; APWNSATL; PWNSATL;
VAPWNSAAL; APWNSAAL; PWNSAAL;
VAPWNAAAL; APWNAAAL; GAVPDLAWL;
GAVPDLATL
and
PWNAAAL.

In an alternative embodiment, the peptide amino acid sequence may comprise any sequence selected from the group comprising:

LNPSVAATI; NPSVAATI; PSVAATI;
VAPWNAATI; APWNAATI; PWNAATI;
VAPWNSATI; APWNSATI; PWNSATI;
VAPWNSAAI; APWNSAAI; PWNSAAI;
VAPWNSAAI; APWNSAAI;
and
PWNSAAI.

The peptide amino acid sequence may comprise any sequence selected from the group comprising:

LNPSVAATL; NPSVAATL; PSVAATL;
LNPSVAAAL; NPSVAAAL; PSVAAAL;
LNPSVAASL; NPSVAASL; PSVAASL;
LNPSVAAWL; NPSVAAWL;
and
PSVAAWL.

The peptide amino acid sequence may comprise any sequence selected from the group comprising:

LNPSVAATI; NPSVAATI; PSVAATI;
LNPSVAAAI; NPSVAAAI; PSVAAAI;
LNPSVAASI; NPSVAASI; PSVAASI;
LNPSVAAWI; NPSVAAWI;
and
PSVAAWI.

The peptide amino acid sequence may comprise any sequence selected from the group comprising: IVDLMCHATF; VIDAMCHATL; AANVMAASL; and FLSDVPVATL.

The peptide may comprise or consist of the amino acid sequence IVDLMCHATF (SEQ ID NO: 1). Alternatively, the peptide may comprise or consist of the amino acid sequence VIDAMCHATL. Alternatively, the peptide may comprise or consist of the amino acid sequence LNPSVAATL.

The peptide may comprise or consist of the amino acid sequence XⁿHATX^{y} wherein Xⁿ is an amino acid sequence of between 5 and 11 residues and wherein X^{y} is any amino acid, L or F. In one embodiment, the peptide may comprise or consist of the amino acid sequence XⁿHATF wherein Xⁿ is an amino acid sequence of between 5 and 11 residues. In another embodiment, the peptide may comprise or consist of the amino acid sequence XⁿHATL wherein Xⁿ is an amino acid sequence of between 5 and 11 residues. In one embodiment, the peptide comprises or consists of the sequence NAPLVHATL.

In one embodiment, the peptide is not full-length exportin-1.

The peptide may be between about 8 and about 12 amino acid residues in length. The peptide may be between about 8 and about 15 amino acid residues in length. The peptide may be at least 8 amino acid residues in length. The peptide may be 9 amino acid residues in length.

In one embodiment, the C terminus of the peptide ends in the sequence ATL, or HATL. In one embodiment wherein the peptide comprises the sequence NAPLVHATL, the N-terminus of the peptide may comprise one or more additional amino acid residues. In one embodiment wherein the peptide comprises the sequence NAPLVHATL, the N-terminus of the peptide may comprise between 1 and 6 additional amino acid residues.

The invention advantageously provides that MHC class I specific peptides can be utilized to drive activation and expansion of NK cells expressing specific inhibitory or activating KIR and that this property can be exploited to generate peptide specific protocols for NK cell therapy against cells overexpressing exportin-1, such as cancer cells.

According to another aspect of the invention, there is provided a complex comprising an MHC class I molecule and the peptide according to the invention.

In one embodiment, the complex comprises an MHC class I molecule and a peptide comprising or consisting of the sequence NAPLVHATL.

The MHC class I molecule may comprise an MHC class I truncated at the stem region of the α3 domain. The MHC class I molecule may comprise HLA-C, or part thereof. The HLA-C may be a group 1 HLA-C (e.g. HLA-C with lysine at residue 80 of the alpha helix of the MHC class I heavy chain). The MHC class I molecule may comprise HLA-C*01. The HLA-C*01 may be HLA-C*0102. The MHC class I molecule may comprise HLA-C*03. The HLA-C*03 may be HLA-C*0304. The MHC class I molecule may comprise any one of HLA-C*01, HLA-C*08, HLA-C*12 HLA-C*16, HLA-C*18, or HLA-C*14. In one embodiment the MHC class I molecule may comprise any one of HLA-C*0102, HLA-C*0801, HLA-C*1201 HLA-C*1601, HLA-C*1801, or HLA-C*1402.

The complex may comprise a fusion protein of the MHC class I molecule and the peptide according to the invention. The complex may comprise a fusion protein of the MHC class I molecule HLA-C*01 and the peptide according to the invention. The HLA-C*01 may be HLA-C*0102. The complex may comprise a fusion protein of the MHC class I molecule HLA-C*03 and the peptide according to the invention. The HLA-C*03 may be HLA-C*0304. In another embodiment, the complex may comprise a fusion protein of the MHC class I molecule of any one of HLA-C*01, HLA-C*08, HLA-C*12, HLA-C*16, HLA-C*18, or HLA-C*14 and the peptide according to the invention. In another embodiment, the complex may comprise a fusion protein of the MHC class I molecule of any one of HLA-C*0102, HLA-C*0801, HLA-C*1201, HLA-C*1601, HLA-C*1801, or HLA-C*1402 and the peptide according to the invention. The complex may be an isolated complex.

According to another aspect of the invention, there is provided a vesicle comprising the complex of the invention.

The vesicle may be an exosome. The vesicle may be isolated. The exosome may be isolated from a cell arranged to express an MHC class I molecule, or part thereof, and the peptide according to the invention. The exosome may be isolated from a cell arranged to express MHC class I molecule HLA-C, or part thereof, and the peptide according to the invention. The exosome may be isolated from a cell arranged to express MHC class I molecule HLA-C*01 and the peptide according to the invention. The HLA-C*01 may be HLA-C*0102. The exosome may be isolated from a cell arranged to express MHC class I molecule HLA-C*03 and the peptide according to the invention. The HLA-C*03 may be HLA-C*0304. The exosome may be isolated from a cell arranged to express MHC class I molecule of any one of HLA-C*01, HLA-C*08, HLA-C*12, HLA-C*16, HLA-C*18, or HLA-C*14 and the peptide according to the invention. Alternatively, the exosome may be isolated from a cell arranged to express MHC class I molecule of any one of HLA-C*0102, HLA-C*0801, HLA-C*1201, HLA-C*1601, HLA-C*1801, or HLA-C*1402 and the peptide according to the invention.

According to another aspect of the invention, there is provided an activated NK cell, wherein the NK cell expresses KIR2DS2 receptor, and wherein the NK cell is activated by exposure to the peptide according to the invention.

The NK cell may be mammalian. The NK cell may be human. The exposure of the NK cell to the peptide to activate the NK cell may be in vitro, for example in a cell culture. The exposure of the NK cell to the peptide to activate the NK cell may cause expansion of the NK cell into multiple NK cells having specificity to specific peptides, such as the peptide according to the invention, or related peptides. One or more cytokines may additionally be added to the NK cells to aid activation and/or expansion.

According to another aspect of the invention, there is provided a nucleic acid comprising a sequence encoding a peptide according to the invention.

The nucleic acid may be a plasmid vector for vaccination. The nucleic acid may comprise DNA or RNA. The nucleic acid may comprise viral nucleic acid. The nucleic acid may comprise a viral vector. The nucleic acid may be a vector. The nucleic acid may comprise Adeno associated virus plasmid DNA.

The nucleic acid may further comprise a sequence encoding an MHC class I molecule. The nucleic acid may comprise a sequence encoding an MHC class I molecule and a peptide comprising or consisting of the sequence NAPLVHATL. The MHC class I molecule may comprise HLA-C, or part thereof. The MHC class I molecule may comprise HLA-C*01. The HLA-C*01 may be HLA-C*0102. The MHC class I molecule may comprise HLA-C*03. The HLA-C*03 may be HLA-C*0304. The MHC class I molecule may comprise any one of HLA-C*01, HLA-C*08, HLA-C*12, HLA-C*16, HLA-C*18, or HLA-C*14. In one embodiment, the MHC class I molecule may comprise any one of HLA-C*0102, HLA-C*0801, HLA-C*1201, HLA-C*1601, HLA-C*1801, or HLA-C*1402. The nucleic acid may comprise a viral promoter, such as SV40 promoter, Rous Sarcoma Virus (RSV) promoter, or cytomegalovirus (CMV) immediate early promoter. The nucleic acid may comprise a sequence encoding Mason-Pfizer monkey virus (MPV)-CTE with or without rev. The nucleic acid may comprise an enhancer sequence. The nucleic acid may comprise a synthetic intron. The nucleic acid may comprise an adenovirus tripartite leader (TPL) sequence. The nucleic acid may comprise a N-terminal ubiquitin signal for targeting the MHC class I pathway. The nucleic acid may encode Adenovirus E3/19K glycoprotein. The nucleic acid may encode a 2A sequence for control of splicing, for example T2A (Thosea asigna virus 2A: T2A), F2A (foot and mouth disease virus 2A) equine rhinitis A virus (E2A), or porcine teschovirus-1 (P2A). The nucleic acid may encode E19/3K, the peptide of the invention, T2A, and a HLA-C, or part thereof. The nucleic acid may encode E19/3K:Peptide:T2A:HLA-C in the orientation provided.

According to another aspect of the invention, there is provided a virus comprising the nucleic acid according to the invention.

The virus may be selected from any of the group comprising adenovirus; Adeno associated virus; Pox viruses e.g. vaccinia such as MVA; Alpha viruses e.g. Semliki forest virus; Lentivirus; Retrovirus; Oncolytic virus e.g. reovirus. The virus may be attenuated.

Reference to a virus herein may also refer to a virus like particle (VLP).

According to another aspect of the invention, there is provided a dendritic cell expressing, or capable of expressing, the complex according to the invention.

The dendritic cell may comprise nucleic acid encoding the complex according to the invention. The dendritic cell may be autologous for the patient. For example, dendritic cell may be harvested from a patient and transformed with nucleic acid encoding the complex according to the invention. Transformants, or generations thereof, may then be returned back to the patient for treatment or prophylaxis (e.g. immunisation).

According to another aspect of the invention, there is provided an immunogenic composition comprising one or more of:
the peptide according to the invention;
the complex according to the invention;
the vesicle according to the invention;
the dendritic cell according to the invention;
the nucleic acid according to the invention; and
the virus according to the invention.

According to another aspect of the invention, there is provided an immunogenic composition comprising the peptide according to the invention.

According to another aspect of the invention, there is provided an immunogenic composition comprising the complex according to the invention.

According to another aspect of the invention, there is provided an immunogenic composition comprising the vesicle according to the invention.

According to another aspect of the invention, there is provided an immunogenic composition comprising the dendritic cell according to the invention.

According to another aspect of the invention, there is provided an immunogenic composition comprising the nucleic acid according to the invention.

According to another aspect of the invention, there is provided an immunogenic composition comprising the virus according to the invention.

The immunogenic composition may comprise a carrier, such as a pharmaceutical acceptable carrier. The carrier may comprise a buffer.

According to another aspect of the invention, there is provided a method of treatment or prophylaxis of cancer, wherein the cancer overexpresses exportin-1, the method comprising the administration of:
the peptide according to the invention;
the immunogenic composition according to the invention;
the complex according to the invention;
the vesicle according to the invention;
the dendritic cell according to the invention;
the activated NK cell according to the invention;
the nucleic acid according to the invention; or
the virus according to the invention.

According to another aspect of the invention, there is provided a method of treatment or prophylaxis of cancer, wherein the cancer overexpresses exportin-1, the method comprising the administration of the peptide according to the invention.

According to another aspect of the invention, there is provided a method of treatment or prophylaxis of cancer, wherein the cancer overexpresses exportin-1, the method comprising the administration of the immunogenic composition according to the invention.

According to another aspect of the invention, there is provided a method of treatment or prophylaxis of cancer, wherein the cancer overexpresses exportin-1, the method comprising the administration of the complex according to the invention.

According to another aspect of the invention, there is provided a method of treatment or prophylaxis of cancer, wherein the cancer overexpresses exportin-1, the method comprising the administration of the vesicle according to the invention.

According to another aspect of the invention, there is provided a method of treatment or prophylaxis of cancer, wherein the cancer overexpresses exportin-1, the method comprising the administration of the dendritic cell according to the invention.

According to another aspect of the invention, there is provided a method of treatment or prophylaxis of cancer, wherein the cancer overexpresses exportin-1, the method comprising the administration of the activated NK cell according to the invention.

According to another aspect of the invention, there is provided a method of treatment or prophylaxis of cancer, wherein the cancer overexpresses exportin-1, the method comprising the administration of the nucleic acid according to the invention.

According to another aspect of the invention, there is provided a method of treatment or prophylaxis of cancer, wherein the cancer overexpresses exportin-1, the method comprising the administration of the virus according to the invention.

The activated NK cells may be provided in the amount of at least 1×10⁷ cells per kg of body weight. The activated NK cells may be provided in the amount of between about 1×10⁷ and 5×10⁹ cells per kg of body weight. The activated NK cells may be provided in the amount of between about 1×10⁷ and 1×10⁹ cells per kg of body weight.

The treatment or prophylaxis may additionally comprise the administration of one or more cytokines. The cytokine(s) may be administered in the same composition as the peptide of the invention. The administration may be intravenously. In an embodiment where cancer is to be treated, the administration may be directly into the artery supplying the cancer.

According to another aspect of the invention, there is provided an agent for use in the prophylaxis or treatment of cancer, wherein the cancer overexpresses exportin-1, the agent comprising or consisting of:
the peptide according to the invention;
the immunogenic composition according to the invention;
the complex according to the invention;
the vesicle according to the invention;
the dendritic cell according to the invention;
the activated NK cell according to the invention;
the nucleic acid according to the invention; or
the virus according to the invention.

According to another aspect of the invention, there is provided an agent for use in the prophylaxis or treatment of cancer, wherein the cancer overexpresses exportin-1, the agent comprising or consisting of the peptide according to the invention.

According to another aspect of the invention, there is provided an agent for use in the prophylaxis or treatment of cancer, wherein the cancer overexpresses exportin-1, the agent comprising or consisting of the immunogenic composition according to the invention.

According to another aspect of the invention, there is provided an agent for use in the prophylaxis or treatment of cancer, wherein the cancer overexpresses exportin-1, the agent comprising or consisting of the complex according to the invention.

According to another aspect of the invention, there is provided an agent for use in the prophylaxis or treatment of cancer, wherein the cancer overexpresses exportin-1, the agent comprising or consisting of the vesicle according to the invention.

According to another aspect of the invention, there is provided an agent for use in the prophylaxis or treatment of cancer, wherein the cancer overexpresses exportin-1, the agent comprising or consisting of the dendritic cell according to the invention.

According to another aspect of the invention, there is provided an agent for use in the prophylaxis or treatment of cancer, wherein the cancer overexpresses exportin-1, the agent comprising or consisting of the activated NK cell according to the invention;

According to another aspect of the invention, there is provided an agent for use in the prophylaxis or treatment of cancer, wherein the cancer overexpresses exportin-1, the agent comprising or consisting of the nucleic acid according to the invention.

According to another aspect of the invention, there is provided an agent for use in the prophylaxis or treatment of cancer, wherein the cancer overexpresses exportin-1, the agent comprising or consisting of the virus according to the invention.

According to another aspect of the invention, there is provided a method of treatment or prophylaxis of a patient for cancer, wherein the cancer overexpresses exportin-1, the method comprising determining if a patient produces KIR2DS2-expressing NK cells and determining if the cancer overexpresses exportin-1;
wherein if the patient produces KIR2DS2-expressing NK cells and the cancer overexpresses exportin-1, administering:
   the peptide according to the invention;
   the immunogenic composition according to the invention;
   the complex according to the invention;
   the vesicle according to the invention;
   the dendritic cell according to the invention;
   the nucleic acid according to the invention; or
   the virus according to the invention; and
optionally wherein if the patient does not produce KIR2DS2-expressing NK cells, administering to the patient the activated NK cells according to the invention.

According to another aspect of the invention, there is provided a method of treatment or prophylaxis of a patient for cancer, wherein the cancer expresses exportin-1, the method comprising determining if a patient produces KIR2DS2-expressing NK cells and determining if the cancer overexpresses exportin-1;
wherein if the patient produces KIR2DS2-expressing NK cells and the cancer overexpresses exportin-1, administering the peptide according to the invention; and optionally wherein if the patient does not produce KIR2DS2-expressing NK cells, administering to the patient the activated NK cells according to the invention.

According to another aspect of the invention, there is provided a method of treatment or prophylaxis of a patient for cancer, wherein the cancer overexpresses exportin-1, the method comprising determining if a patient produces KIR2DS2-expressing NK cells and determining if the cancer overexpresses exportin-1;
wherein if the patient produces KIR2DS2-expressing NK cells and the cancer overexpresses exportin-1, administering the immunogenic composition according to the invention; and optionally wherein if the patient does not produce KIR2DS2-expressing NK cells, administering to the patient the activated NK cells according to the invention.

According to another aspect of the invention, there is provided a method of treatment or prophylaxis of a patient for cancer, wherein the cancer overexpresses exportin-1, the method comprising determining if a patient produces KIR2DS2-expressing NK cells and determining if the cancer overexpresses exportin-1;
wherein if the patient produces KIR2DS2-expressing NK cells and the cancer overexpresses exportin-1, administering the complex according to the invention; and optionally wherein if the patient does not produce KIR2DS2-expressing NK cells, administering to the patient the activated NK cells according to the invention.

According to another aspect of the invention, there is provided a method of treatment or prophylaxis of a patient for cancer, wherein the cancer overexpresses exportin-1, the method comprising determining if a patient produces KIR2DS2-expressing NK cells and determining if the cancer overexpresses exportin-1;
wherein if the patient produces KIR2DS2-expressing NK cells and the cancer overexpresses exportin-1, administering the vesicle according to the invention; and optionally wherein if the patient does not produce KIR2DS2-expressing NK cells, administering to the patient the activated NK cells according to the invention.

According to another aspect of the invention, there is provided a method of treatment or prophylaxis of a patient for cancer, wherein the cancer overexpresses exportin-1, the method comprising determining if a patient produces KIR2DS2-expressing NK cells and determining if the cancer overexpresses exportin-1;
wherein if the patient produces KIR2DS2-expressing NK cells and the cancer overexpresses exportin-1, administering the dendritic cell according to the invention; and optionally wherein if the patient does not produce KIR2DS2-expressing NK cells, administering to the patient the activated NK cells according to the invention.

According to another aspect of the invention, there is provided a method of treatment or prophylaxis of a patient for cancer, wherein the cancer overexpresses exportin-1, the method comprising determining if a patient produces KIR2DS2-expressing NK cells and determining if the cancer overexpresses exportin-1;
wherein if the patient produces KIR2DS2-expressing NK cells and the cancer overexpresses exportin-1, administering the nucleic acid according to the invention; and optionally wherein if the patient does not produce KIR2DS2-expressing NK cells, administering to the patient the activated NK cells according to the invention.

According to another aspect of the invention, there is provided a method of treatment or prophylaxis of a patient for cancer, wherein the cancer overexpresses exportin-1, the method comprising determining if a patient produces KIR2DS2-expressing NK cells and determining if the cancer overexpresses exportin-1;
wherein if the patient produces KIR2DS2-expressing NK cells and the cancer overexpresses exportin-1, administering the virus according to the invention; and optionally wherein if the patient does not produce KIR2DS2-expressing NK cells, administering to the patient the activated NK cells according to the invention.

According to another aspect of the invention, there is provided a method of treatment or prophylaxis of a patient for cancer, wherein the cancer overexpresses exportin-1, the method comprising determining if a patient produces a ligand for KIR2DS2 and determining if the cancer overexpresses exportin-1;
wherein if the patient produces a ligand for KIR2DS2 and the cancer overexpresses exportin-1, administering:
   the peptide according to the invention;
   the immunogenic composition according to the invention;
   the complex according to the invention;
   the vesicle according to the invention;
   the dendritic cell according to the invention;
   the nucleic acid according to the invention; or
   the virus according to the invention; and
optionally wherein if the patient does not produce a ligand for KIR2DS2, administering to the patient the activated NK cells according to the invention.

According to another aspect of the invention, there is provided a method of treatment or prophylaxis of a patient for cancer, wherein the cancer overexpresses exportin-1, the method comprising determining if a patient produces a ligand for KIR2DS2 and determining if the cancer overexpresses exportin-1;
wherein if the patient produces a ligand for KIR2DS2 and the cancer overexpresses exportin-1, administering the peptide according to the invention; and
optionally wherein if the patient does not produce a ligand for KIR2DS2, administering to the patient the activated NK cells according to the invention.

According to another aspect of the invention, there is provided a method of treatment or prophylaxis of a patient for cancer, wherein the cancer overexpresses exportin-1, the method comprising determining if a patient produces a ligand for KIR2DS2 and determining if the cancer overexpresses exportin-1;
wherein if the patient produces a ligand for KIR2DS2 and the cancer overexpresses exportin-1, administering the immunogenic composition according to the invention; and
optionally wherein if the patient does not produce a ligand for KIR2DS2, administering to the patient the activated NK cells according to the invention.

According to another aspect of the invention, there is provided a method of treatment or prophylaxis of a patient for cancer, wherein the cancer overexpresses exportin-1, the method comprising determining if a patient produces a ligand for KIR2DS2 and determining if the cancer overexpresses exportin-1;
wherein if the patient produces a ligand for KIR2DS2 and the cancer overexpresses exportin-1, administering the complex according to the invention; and
optionally wherein if the patient does not produce a ligand for KIR2DS2, administering to the patient the activated NK cells according to the invention.

According to another aspect of the invention, there is provided a method of treatment or prophylaxis of a patient for cancer, wherein the cancer overexpresses exportin-1, the method comprising determining if a patient produces a ligand for KIR2DS2 and determining if the cancer overexpresses exportin-1;
wherein if the patient produces a ligand for KIR2DS2 and the cancer overexpresses exportin-1, administering the vesicle according to the invention; and
optionally wherein if the patient does not produce a ligand for KIR2DS2, administering to the patient the activated NK cells according to the invention.

According to another aspect of the invention, there is provided a method of treatment or prophylaxis of a patient for cancer, wherein the cancer overexpresses exportin-1, the method comprising determining if a patient produces a ligand for KIR2DS2 and determining if the cancer overexpresses exportin-1;
wherein if the patient produces a ligand for KIR2DS2 and the cancer overexpresses exportin-1, administering the dendritic cell according to the invention; and
optionally wherein if the patient does not produce a ligand for KIR2DS2, administering to the patient the activated NK cells according to the invention.

According to another aspect of the invention, there is provided a method of treatment or prophylaxis of a patient for cancer, wherein the cancer overexpresses exportin-1, the method comprising determining if a patient produces a ligand for KIR2DS2 and determining if the cancer overexpresses exportin-1;
wherein if the patient produces a ligand for KIR2DS2 and the cancer overexpresses exportin-1, administering the nucleic acid according to the invention; and
optionally wherein if the patient does not produce a ligand for KIR2DS2, administering to the patient the activated NK cells according to the invention.

According to another aspect of the invention, there is provided a method of treatment or prophylaxis of a patient for cancer, wherein the cancer overexpresses exportin-1, the method comprising determining if a patient produces a ligand for KIR2DS2 and determining if the cancer overexpresses exportin-1;
wherein if the patient produces a ligand for KIR2DS2 and the cancer overexpresses exportin-1, administering the virus according to the invention; and
optionally wherein if the patient does not produce a ligand for KIR2DS2, administering to the patient the activated NK cells according to the invention.

According to another aspect of the invention, there is provided a method of selecting a patient for treatment or prophylaxis with an agent arranged to activate NK cell mediated protection from cancer, wherein the cancer overexpresses exportin-1, wherein the agent is selected from:
the peptide according to the invention;
the immunogenic composition according to the invention;
the complex according to the invention;
the vesicle according to the invention;
the nucleic acid according to the invention; or
the virus according to the invention;
the method comprising the step of determining if the patient produces KIR2DS2-expressing NK cells and determining if the cancer overexpresses exportin-1,
wherein a patient is selected for the treatment or prophylaxis with the agent if they produce KIR2DS2-expressing NK cells and the cancer overexpresses exportin-1; and optionally wherein a patient not producing KIR2DS2-expressing NK cells and the cancer does not overexpress exportin-1, the patient is not selected for the treatment or prophylaxis with the agent, and/or is selected for an alternative cancer treatment.

According to another aspect of the invention, there is provided a method of selecting a patient for treatment or prophylaxis with an agent arranged to activate NK cell mediated protection from cancer, wherein the cancer overexpresses exportin-1, wherein the agent is the peptide according to the invention;
the method comprising the step of determining if the patient produces KIR2DS2-expressing NK cells and determining if the cancer overexpresses exportin-1,
wherein a patient is selected for the treatment or prophylaxis with the agent if they produce KIR2DS2-expressing NK cells and the cancer overexpresses exportin-1; and optionally wherein a patient not producing KIR2DS2-expressing NK cells and the cancer does not overexpress exportin-1, the patient is not selected for the treatment or prophylaxis with the agent, and/or is selected for an alternative cancer treatment.

According to another aspect of the invention, there is provided a method of selecting a patient for treatment or prophylaxis with an agent arranged to activate NK cell mediated protection from cancer, wherein the cancer overexpresses exportin-1, wherein the agent is the immunogenic composition according to the invention;
the method comprising the step of determining if the patient produces KIR2DS2-expressing NK cells and determining if the cancer overexpresses exportin-1,
wherein a patient is selected for the treatment or prophylaxis with the agent if they produce KIR2DS2-expressing NK cells and the cancer overexpresses exportin-1; and optionally wherein a patient not producing KIR2DS2-expressing NK cells and the cancer does not overexpress exportin-1, the patient is not selected for the treatment or prophylaxis with the agent, and/or is selected for an alternative cancer treatment.

According to another aspect of the invention, there is provided a method of selecting a patient for treatment or prophylaxis with an agent arranged to activate NK cell mediated protection from cancer, wherein the cancer overexpresses exportin-1, wherein the agent is the complex according to the invention;
the method comprising the step of determining if the patient produces KIR2DS2-expressing NK cells and determining if the cancer overexpresses exportin-1,
wherein a patient is selected for the treatment or prophylaxis with the agent if they produce KIR2DS2-expressing NK cells and the cancer overexpresses exportin-1; and optionally wherein a patient not producing KIR2DS2-expressing NK cells and the cancer does not overexpress exportin-1, the patient is not selected for the treatment or prophylaxis with the agent, and/or is selected for an alternative cancer treatment.

According to another aspect of the invention, there is provided a method of selecting a patient for treatment or prophylaxis with an agent arranged to activate NK cell mediated protection from cancer, wherein the cancer overexpresses exportin-1, wherein the agent is the vesicle according to the invention;
the method comprising the step of determining if the patient produces KIR2DS2-expressing NK cells and determining if the cancer overexpresses exportin-1,
wherein a patient is selected for the treatment or prophylaxis with the agent if they produce KIR2DS2-expressing NK cells and the cancer overexpresses exportin-1; and optionally wherein a patient not producing KIR2DS2-expressing NK cells and the cancer does not overexpress exportin-1, the patient is not selected for the treatment or prophylaxis with the agent, and/or is selected for an alternative cancer treatment.

According to another aspect of the invention, there is provided a method of selecting a patient for treatment or prophylaxis with an agent arranged to activate NK cell mediated protection from cancer, wherein the cancer overexpresses exportin-1, wherein the agent is the nucleic acid according to the invention;
the method comprising the step of determining if the patient produces KIR2DS2-expressing NK cells and determining if the cancer overexpresses exportin-1,
wherein a patient is selected for the treatment or prophylaxis with the agent if they produce KIR2DS2-expressing NK cells and the cancer overexpresses exportin-1; and optionally wherein a patient not producing KIR2DS2-expressing NK cells and the cancer does not overexpress exportin-1, the patient is not selected for the treatment or prophylaxis with the agent, and/or is selected for an alternative cancer treatment.

According to another aspect of the invention, there is provided a method of selecting a patient for treatment or prophylaxis with an agent arranged to activate NK cell mediated protection from cancer, wherein the cancer overexpresses exportin-1, wherein the agent is the virus according to the invention;
the method comprising the step of determining if the patient produces KIR2DS2-expressing NK cells and determining if the cancer overexpresses exportin-1,
wherein a patient is selected for the treatment or prophylaxis with the agent if they produce KIR2DS2-expressing NK cells and the cancer overexpresses exportin-1; and optionally wherein a patient not producing KIR2DS2-expressing NK cells and the cancer does not overexpress exportin-1, the patient is not selected for the treatment or prophylaxis with the agent, and/or is selected for an alternative cancer treatment.

According to another aspect of the invention, there is provided a method of selecting a patient for treatment or prophylaxis with an agent arranged to activate NK cell mediated protection from cancer, wherein the cancer overexpresses exportin-1, wherein the agent is selected from:
the peptide according to the invention;
the immunogenic composition according to the invention;
the complex according to the invention;
the vesicle according to the invention;
the nucleic acid according to the invention; or
the virus according to the invention;
the method comprising the step of determining if the patient produces a ligand for KIR2DS2 and determining if the cancer overexpresses exportin-1,
wherein a patient is selected for the treatment or prophylaxis with the agent if they produce a ligand for KIR2DS2 and the cancer overexpresses exportin-1; and optionally wherein a patient not producing a ligand for KIR2DS2 is not selected for the treatment or prophylaxis with the agent, and/or is selected for an alternative cancer treatment.

According to another aspect of the invention, there is provided a method of selecting a patient for treatment or prophylaxis with an agent arranged to activate NK cell mediated protection from cancer, wherein the cancer overexpresses exportin-1, wherein the agent is the peptide according to the invention;
the method comprising the step of determining if the patient produces a ligand for KIR2DS2 and determining if the cancer overexpresses exportin-1,
wherein a patient is selected for the treatment or prophylaxis with the agent if they produce a ligand for KIR2DS2 and the cancer overexpresses exportin-1; and optionally wherein a patient not producing a ligand for KIR2DS2 is not selected for the treatment or prophylaxis with the agent, and/or is selected for an alternative cancer treatment.

According to another aspect of the invention, there is provided a method of selecting a patient for treatment or prophylaxis with an agent arranged to activate NK cell mediated protection from cancer, wherein the cancer overexpresses exportin-1, wherein the agent is the immunogenic composition according to the invention;
the method comprising the step of determining if the patient produces a ligand for KIR2DS2 and determining if the cancer overexpresses exportin-1,
wherein a patient is selected for the treatment or prophylaxis with the agent if they produce a ligand for KIR2DS2 and the cancer overexpresses exportin-1; and optionally wherein a patient not producing a ligand for KIR2DS2 is not selected for the treatment or prophylaxis with the agent, and/or is selected for an alternative cancer treatment.

According to another aspect of the invention, there is provided a method of selecting a patient for treatment or prophylaxis with an agent arranged to activate NK cell mediated protection from cancer, wherein the cancer overexpresses exportin-1, wherein the agent is the complex according to the invention;
the method comprising the step of determining if the patient produces a ligand for KIR2DS2 and determining if the cancer overexpresses exportin-1,
wherein a patient is selected for the treatment or prophylaxis with the agent if they produce a ligand for KIR2DS2 and the cancer overexpresses exportin-1; and optionally wherein a patient not producing a ligand for KIR2DS2 is not selected for the treatment or prophylaxis with the agent, and/or is selected for an alternative cancer treatment.

According to another aspect of the invention, there is provided a method of selecting a patient for treatment or prophylaxis with an agent arranged to activate NK cell mediated protection from cancer, wherein the cancer overexpresses exportin-1, wherein the agent is the vesicle according to the invention;
the method comprising the step of determining if the patient produces a ligand for KIR2DS2 and determining if the cancer overexpresses exportin-1,
wherein a patient is selected for the treatment or prophylaxis with the agent if they produce a ligand for KIR2DS2 and the cancer overexpresses exportin-1; and optionally wherein a patient not producing a ligand for KIR2DS2 is not selected for the treatment or prophylaxis with the agent, and/or is selected for an alternative cancer treatment.

According to another aspect of the invention, there is provided a method of selecting a patient for treatment or prophylaxis with an agent arranged to activate NK cell mediated protection from cancer, wherein the cancer overexpresses exportin-1, wherein the agent is the nucleic acid according to the invention;
the method comprising the step of determining if the patient produces a ligand for KIR2DS2 and determining if the cancer overexpresses exportin-1,
wherein a patient is selected for the treatment or prophylaxis with the agent if they produce a ligand for KIR2DS2 and the cancer overexpresses exportin-1; and optionally wherein a patient not producing a ligand for KIR2DS2 is not selected for the treatment or prophylaxis with the agent, and/or is selected for an alternative cancer treatment.

According to another aspect of the invention, there is provided a method of selecting a patient for treatment or prophylaxis with an agent arranged to activate NK cell mediated protection from cancer, wherein the cancer overexpresses exportin-1, wherein the agent is the virus according to the invention;
the method comprising the step of determining if the patient produces a ligand for KIR2DS2 and determining if the cancer overexpresses exportin-1,
wherein a patient is selected for the treatment or prophylaxis with the agent if they produce a ligand for KIR2DS2 and the cancer overexpresses exportin-1; and optionally wherein a patient not producing a ligand for KIR2DS2 is not selected for the treatment or prophylaxis with the agent, and/or is selected for an alternative cancer treatment.

The ligand for KIR2DS2 may comprise a Group I HLA-C allele encoded ligand. The ligand for KIR2DS2 may comprise HLA-A11.

The method for selecting a patient may further comprise administering the agent to the selected patient.

The alternative cancer treatment may comprise administering to the patient, the activated NK cell according to the invention.

Determining if a patient produces KIR2DS2-expressing NK cells may comprise providing a sample of patient blood or blood plasma and detecting KIR2DS2-expressing NK cells in the blood or blood plasma. Determining if a patient produces KIR2DS2-expressing NK cells may comprise providing a sample of patient blood or blood plasma and detecting nucleic acid encoding KIR2DS2. The detection may comprise PCR, for example using sequence specific primers (PCR-SSP). The detection may comprise nucleic acid sequencing, such as RNA sequencing. The detection may comprise antibody-mediated detection. The detection may comprise KIR2DS2 ligand-receptor binding mediated detection.

Determining if a patient produces a ligand for KIR2DS2 may comprise providing a sample of patient blood or blood plasma and detecting a ligand for KIR2DS2 in the blood or blood plasma. Determining if a patient produces a ligand for KIR2DS2 may comprise providing a sample of patient blood or blood plasma and detecting nucleic acid encoding a ligand for KIR2DS2. The detection may comprise PCR, for example using sequence specific primers (PCR-SSP). The detection may comprise nucleic acid sequencing, such as RNA sequencing. The detection may comprise antibody-mediated detection. The detection may comprise ligand-receptor binding mediated detection.

Determining if the cancer overexpresses exportin-1 may comprise providing a sample of patient blood or blood plasma, or a biopsy of the cancer, and detecting exportin-1, or nucleic acid encoding exportin-1. The detection may comprise determining the level of exportin-1, or nucleic acid transcripts thereof. The level may be compared to a reference level or score, which represents exportin-1 levels in non-exportin-1 related cancers and/or normal cells of the same tissue.

The cancer may be a cancer capable of being inhibited, suppressed, cured, alleviated or prevented by the action of specific NK cell mediated killing. The cancer may be a cancer capable of being modified by the action of specific NK cell killing.

In one embodiment, the cancer is a cancer selected from the group comprising liver cancer, melanoma, lymphoma, pancreatic cancer, breast cancer, lung cancer, renal cancer, leukaemia and multiple myeloma.

In another embodiment, the cancer is a cancer selected from the group comprising liver cancer, melanoma, leukaemia and multiple myeloma. In another embodiment, the cancer is liver cancer.

In another embodiment, the cancer is a cancer selected from the group comprising lung cancer, osteosarcoma, glioma, pancreatic cancer, ovarian cancer, cervical carcinoma, renal cell carcinoma, oesophageal carcinoma, gastric carcinoma, hepatocellular carcinoma, acute myeloid/lymphoid leukaemia, chronic myeloid/lymphoid leukaemia, mantle cell lymphoma, plasma cell leukaemia and multiple myeloma.

In another embodiment, the cancer is blood cancer, acute myeloid leukaemia, chronic myeloid leukaemia, lymphoma, or multiple myeloma.

The human protein atlas provides that 100% patients express exportin-1 in the cancers of thyroid, lung, carcinoid, colorectal, head and neck, stomach, urothelial, testis, breast, ovarian, melanoma, skin, >80% express exportin-1 in glioma, cervical, pancreatic cancers and lymphoma, and >30% express exportin-1 in endometrial, prostate and liver cancer. 2/9 patients with renal cancer express exportin-1. Expression of exportin-1 is adversely associated with survival in leukaemia, liver and renal cancer.

According to another aspect of the invention, there is provided a method of producing activated NK cells comprising exposing an NK cell expressing KIR2DS2 receptor to a peptide according to the invention.

The NK cell expressing KIR2DS2 receptor may be exposed to a peptide comprising or consisting of the sequence NAPLVHATL.

The method of producing activated NK cells may be in vitro, for example in a cell culture.

According to another aspect of the invention, there is provided a method for activating an NK cell mediated immune response of a patient for recognition of exportin-1 comprising administration of:
the peptide according to the invention;
the complex according to the invention;
the vesicle according to the invention;
the nucleic acid according to the invention; or
the virus according to the invention.

According to another aspect of the invention, there is provided a method for activating an NK cell mediated immune response of a patient for recognition of exportin-1, or a part thereof, comprising administration of the peptide according to the invention.

According to another aspect of the invention, there is provided a method for activating an NK cell mediated immune response of a patient for recognition of exportin-1, or a part thereof, comprising administration of the complex according to the invention.

According to another aspect of the invention, there is provided a method for activating an NK cell mediated immune response of a patient for recognition of exportin-1, or a part thereof, comprising administration of the vesicle according to the invention.

According to another aspect of the invention, there is provided a method for activating an NK cell mediated immune response of a patient for recognition of exportin-1, or a part thereof, comprising administration of the nucleic acid according to the invention.

According to another aspect of the invention, there is provided a method for activating an NK cell mediated immune response of a patient for recognition of exportin-1, or a part thereof, comprising administration of the virus according to the invention.

According to another aspect of the invention, there is provided the use of:
the peptide according to the invention;
the complex according to the invention;
the vesicle according to the invention;
the nucleic acid according to the invention; or
the virus according to the invention,
   for activating an NK cell mediated immune response of a patient for recognition of exportin-1, or a part thereof.

Recognition of exportin-1, or a part thereof, may comprise recognition of cells that overexpress exportin-1, or a part thereof. The recognition of exportin-1 overexpressing cells may further comprise killing of such cells.

The immune response may be protective.

According to another aspect of the invention, there is provided a peptide according to the invention for use as a vaccine. According to another aspect of the invention, there is provided a nucleic acid according to the invention for use as a vaccine.

The vaccine may be a vaccine against exportin-1, or cells that overexpress exportin-1.

The term “overexpresses exportin-1” is understood to mean a cell that has elevated expression, such as an elevated number of transcripts or exportin-1 protein, relative to a normal cell or relative to the expression in an average population of the same cell type. The expression level may be relative to other non-cancerous cells of the same type, optionally from the same patient. Alternatively, the overexpression may be determined by reference to a pre-determined normal expression value for exportin-1. Over expression of exportin-1 may be defined as significantly or statistically greater RNA transcript levels than those described as normal in the Human Protein Atlas (www.proteinatlas.org) and referenced in Uhlen M et al, 2017. A pathology atlas of the human cancer transcriptome. Science. (PubMed: 28818916 DOI: 10.1126/science.aan2507), which is herein incorporated by reference. In particular, a skilled person would readily identify using publically available datasets, an overexpression of exportin-1 in a given cell type.

The term “immunogenic”, when applied to the peptide or composition of the present invention means capable of eliciting an immune response in a human or animal body.

The term “isolated”, when applied to the peptide or complex of the present invention means a peptide or complex: (i) encoded by nucleic acids using recombinant DNA methods; or (ii); synthesized by, for example, chemical synthetic methods; or (iii) separated from naturally-occurring biological materials, and then purified using protein analytical procedures; or (iv) associated with chemical moieties (e.g. peptides, carbohydrates, fatty acids, and the like) other than those associated with the antigenic peptide in its naturally-occurring state; or (v) that do not occur in nature. An isolated peptide or complex of the invention includes a peptide or complex expressed from a nucleotide sequence encoding the peptide or complex, or from a recombinant vector containing a nucleotide sequence encoding the peptide or complex.

The term “protective” means prevention of a disease, a reduced risk of disease infection, transmission and/or progression, reduced severity of disease, a cure of a condition or disease, an alleviation of symptoms, or a reduction in severity of a disease or disease symptoms.

The term “prophylaxis” means prevention of or protective treatment for a disease. The prophylaxis may include a reduced risk of disease infection, transmission and/or progression, or reduced severity of disease.

The term “treatment”, means a cure of a condition or disease, an alleviation of symptoms, or a reduction in severity of a disease or disease symptoms.

The skilled person will understand that optional features of one embodiment or aspect of the invention may be applicable, where appropriate, to other embodiments or aspects of the invention.

Embodiments of the invention will now be described in more detail, by way of example only, with reference to the accompanying drawings.

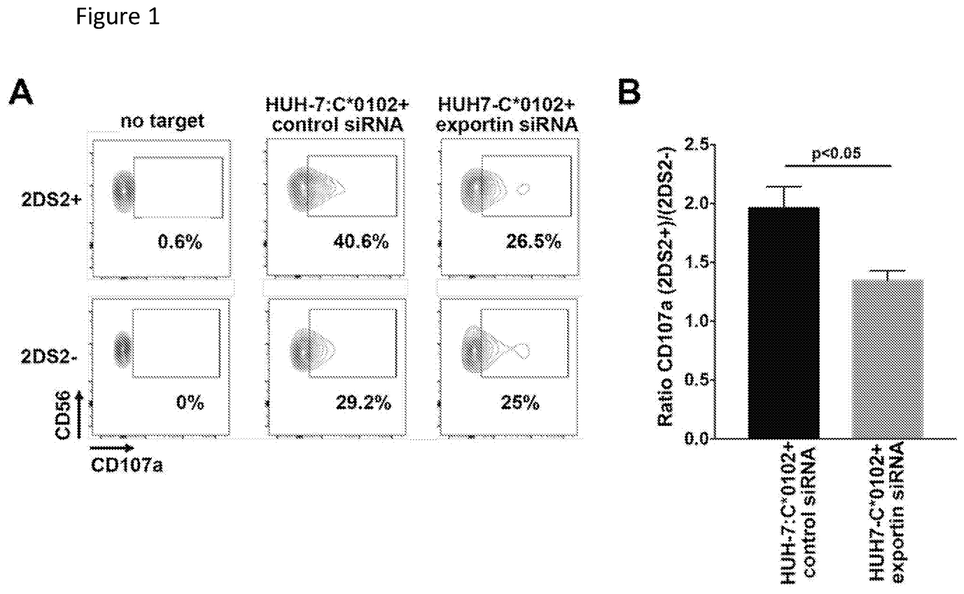

FIG. 1—Exportin-1 is recognised by natural killer cells expressing KIR2DS2.

A flow cytometry plots of CD107a on KIR2DS2+ or KIR2DS2− NK cells are shown when they are incubated in the absence of targets, or in the presence of HUH-7:C*0102 cells transfected with control siRNA or an siRNA that targets exportin1. CD3-CD56+ NK cells are shown.

B, a comparison is made of the degranulation (CD107a expression) of KIR2DS2+ or KIR2DS2− NK cells when cultured with the HUH7-C*0102 cell lines transfected with either control siRNA or siRNA specific for exportin-1. The data are from six experiments and are expressed as the relative ratios of CD107a expression on the KIR2DS2+ and KIR2DS2− NK cells from six experiments.

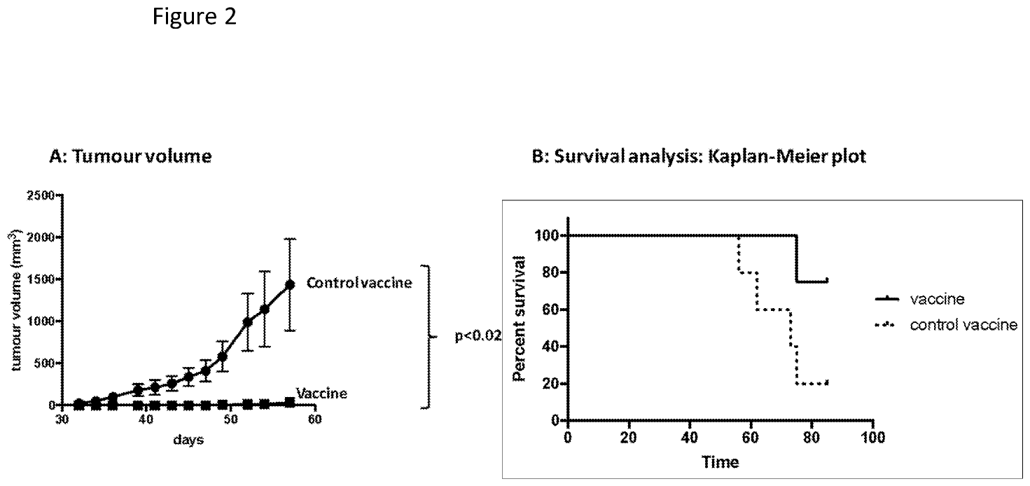

FIG. 2—Vaccination with HLA-C*0102-T2A-E3/19k-IVDVLMCHATF induces an anti-cancer response to HUH-7 cells.

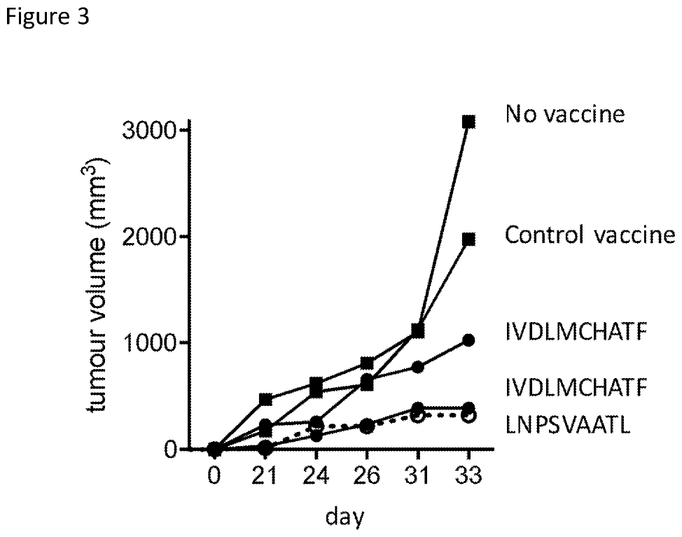

FIG. 3—Vaccination with HLA-C*0102-T2A-E3/19k-LNPSVAATL gives similar results to vaccination with HLA-C*0102-T2A-E3/19k-IVDVLMCHATF.

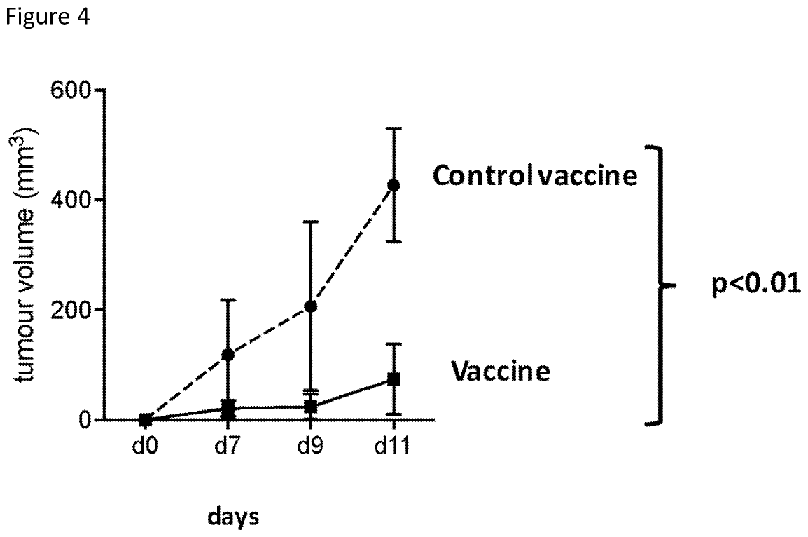

FIG. 4—Vaccination with HLA-C*0102-T2A-E3/19k-IVDVLMCHATF induces an anti-cancer response to lymphoma.

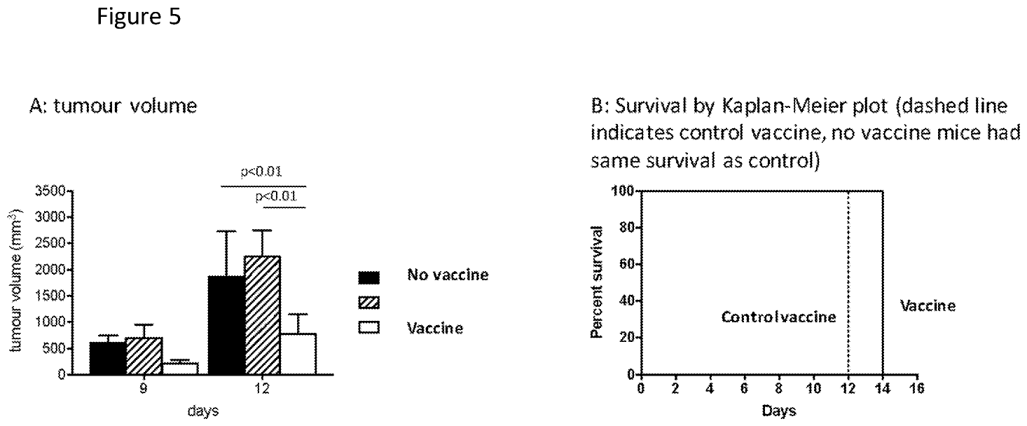

FIG. 5—Vaccination with HLA-C*0102-T2A-E3/19k-IVDVLMCHATF induces an anti-cancer response to melanoma.

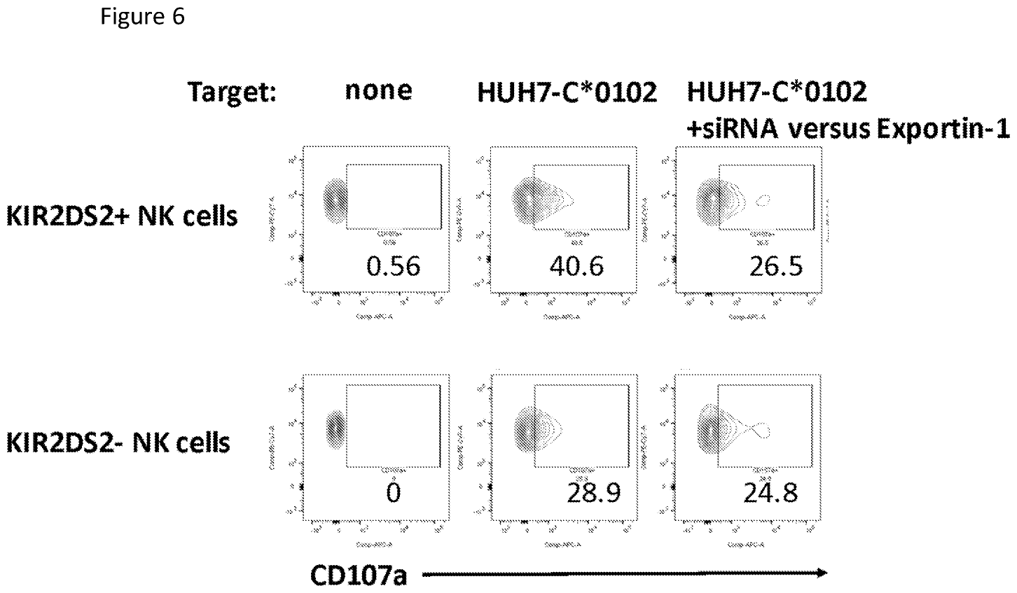

FIG. 6—Knockdown of Exportin-1 decreases killing of HUH-7 cells by KIR2DS2-positive NK cells but not KIR2DS2-negative NK cells.

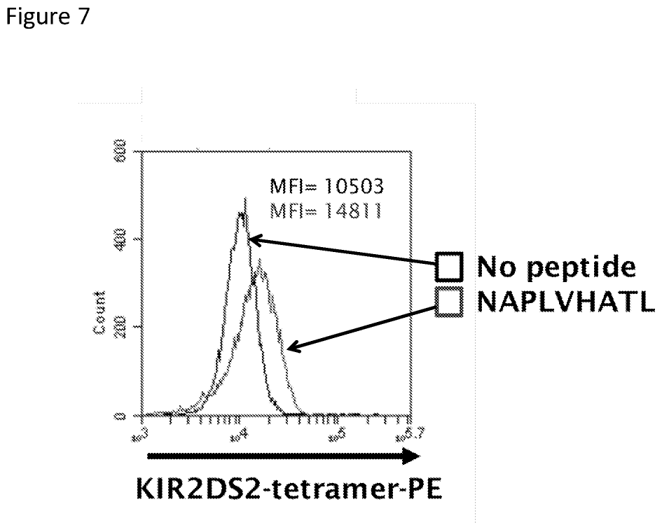

FIG. 7—NAPLVHATL, a peptide derived from Exportin-1 binds KIR2DS2.

### EXAMPLE 1

### Exportin-1 is Recognised by Natural Killer Cells Expressing KIR2DS2

With Reference to FIG. 1, natural killer (NK) cells were incubated with the liver cancer cell line HUH-7 transfected with HLA-C*0102 (HUH-7:C*0102) and then stained for expression of CD107a, a marker of NK cell degranulation/activation. We observed a significant decrease if the HUH-7:C*0102 were transfected with an interfering RNA (siRNA) specific for exportin-1 as compared to a control siRNA.

### EXAMPLE 2

### Vaccination with HLA-C*0102-T2A-E3/19K-IVDVLMCHATF Induces an Anti-Cancer Response to HUH-7 Cells

KIR-transgenic mice were vaccinated with two 50 microgram doses of the DNA vaccine HLA-C*0102-T2A-E3/19k-IVDVLMCHATF, or a control vaccine in which the peptide was mutated to prevent HLA-C binding HLA-C*0102-T2A-E3/19k-IVDVLMCHAAA. Vaccine doses were given one week apart. NOD-SCID-gamma chain knockout (NSG) mice were inoculated with HUH7 cells expressing HLA-C*0102. On days 0 and 21 after tumour challenge NK cells from the vaccinated mice they were infused with purified NK cells from the vaccinated mice. Tumour growth was monitored by measuring and mice sacrificed at a humane endpoint, when the tumour volume exceeds 2000 mm³. Tumour growth was significantly attenuated in the vaccine group (Panel A: tumor volume measurement, p<0.02) and survived significantly longer in the vaccinated group (Panel B: Kaplan-Meier plot, p<0.02) (5 mice per group)

### EXAMPLE 3

### Vaccination with HLA-C*0102-T2A-E3/19K-LNPSVAATL Gives Similar Results to Vaccination with HLA-C*0102-T2A-E3/19K-IVDVLMCHATF

KIR-transgenic mice were vaccinated with two 50 microgram doses of the DNA vaccine HLA-C*0102-T2A-E3/19k-LNPSVAATL or HLA-C*0102-T2A-E3/19k-IVDVLMCHATF, or a control vaccine containing only the HLA-C construct (HLA-C*0102). Tumour growth was then monitored. Each line indicates a different mouse. LNPSVAATL is shown by the dashed line and open circles.

### EXAMPLE 4

### Vaccination with HLA-C*0102-T2A-E3/19K-IVDVLMCHATF Induces an Anti-Cancer Response to Lymphoma

KIR-transgenic mice were vaccinated with two 50 microgram doses of the DNA vaccine HLA-C*0102-T2A-E3/19k-IVDVLMCHATF, or a control vaccine containing only the HLA-C construct (HLA-C*0102) and simultaneously challenged with RMA-S lymphoma cells. Vaccine doses were given on day 0 and day 7. Tumour volume was then monitored. Mice from the vaccinate group had significantly less tumour growth (p<0.01). (3 mice per group)

### EXAMPLE 5

### Vaccination with HLA-C*0102-T2A-E3/19K-IVDVLMCHATF Induces an Anti-Cancer Response to Melanoma

KIR-transgenic mice were vaccinated with two 50 microgram doses of the DNA vaccine HLA-C*0102-T2A-E3/19k-IVDVLMCHATF, or a control vaccine in which the peptide was mutated to prevent HLA-C binding HLA-C*0102-T2A-E3/19k-IVDVLMCHAAA. and simultaneously challenged with B16 melanoma cells. Vaccine doses were given on day 0 and day 7. Tumour volume was then monitored. Results from days 9 and 12 are shown in panel A (p<0.01 vs control vaccine or versus no vaccine) and demonstrated that there is significant attenuation of overall survival in panel B. Mice were sacrificed at a human endpoint when tumour volume exceeded 2000 mm³.

### EXAMPLE 6

### Knockdown of Exportin-1 Decreases Killing of HUH-7 Cells by KIR2DS2-Positive NK Cells but not KIR2DS2-Negative NK Cells

HUH-7-C*0102 cells were transfected with siRNA to reduce expression of Exportin-1. These cells were then tested for killing by KIR2DS2+NK cells or KIR2DS2− NK cells as measured using a CD107a degranulation assay. Knockdown of exportin-1 reduced degranlulation of KIR2DS+NK cells from 40.6% to 26.5%, whereas there was a minimal change for the KIR2DS2− population

### EXAMPLE 7

### NAPLVHATL, a Peptide Derived from Exportin-1 Binds KIR2DS2

721.174 cells were loaded with peptide and then stained using a tetramer of KIR2DS2 conjugated to phycoerythrin. Shown is a flow cytometry histogram plot of the staining of the 721.174 cells in the presence or absence of peptide. Mean fluorescence intensitites are indicated: 14811 for NAPLVHATL versus 10503 for no peptide.

### EXAMPLE 8

### HLA Binding

TABLE 1
Amino acid sequences of NAPLVHATL, LNPSVAATL and
IVDLMCHATF with homology at carboxy-terminal -1 and -2
positions indicated in bold. The Rank score from a NetMHCpan 4.0
Server analysis is shown, with a Rank score of <2 is predictive of
binding to the HLA-C allele indicated.

| Source of | Amino acid | | Rank (Predicted |
|---|---|---|---|
| peptide | sequence | HLA allele | binding stren [symbol] |
| XPO1 | NAPLVHATL | HLA-C*0102 | 0.138 |
| XPO1 | NAPLVHATL | HLA-C*0801 | 0.494 |
| XPO1 | NAPLVHATL | HLA-C*1601 | 0.728 |
| XPO1 | NAPLVHATL | HLA-C*1801 | 1.574 |
| XPO1 | NAPLVHATL | HLA-C*1402 | 0.959 |
| Hepatitis C virus | LNPSVAATL | HLA-C*0102 | 1.812 |
| Dengue virus | IVDLMCHATF | HLA-C*0102 | 3.839 |

Discussion

We have previously described a DNA vaccine in the formation HLA-C:T2A:E3/19k:peptide conformation where peptide is a 5-12 amino acid peptide in the conformation X(n)ATX where n=2-9. We now describe the use of such vaccine using the exemplar HLA-C*0102-T2A-E3/19k-IVDVLMCHATF to activate natural killer (NK) cells expressing the receptor KIR2DS2 as a cancer therapy targeting cancers that express Exportin 1. We have vaccinated KIR-transgenic mice with DNA encoding HLA-C*0102-T2A-E3/19k-IVDVLMCHATF and then adoptively transferred the NK cells from these mice into NOD-SCID-gamma chain deficient mice (NSG). We have challenged these mice with the liver cancer cell line HUH7 expressing HLA-C*0102 (HUH7-C*0102). This has resulted in significant impairment of tumour growth in mice given NK cells from the vaccinated mice, as opposed to mice given NK cells from control vaccinated mice. We have repeated this experiment 3 times with similar results in attenuation of tumour growth. We also show improved survival in the mice given NK cells from vaccinated mice (see FIG. 2B). We also have shown that the vaccine HLA-C*0102-T2A-E3/19k-LNPSVAATL also reduces tumour growth.

We have identified that the tumor cell line HUH7-C*0102 expresses Exportin-1 (XpoI) and that the peptide NAPLVHATL derived from this protein is presented by HLA-C*0102 to NK cells. In vitro assays using human NK cells knock-down of this peptide reduces the killing of the cell line by human NK cells expressing the receptor KIR2DS2, but not by NK cells not expressing the receptor KIR2DS2. Therefore Exportin-1 provides a peptide NAPLVHATL that is recognised by KIR2DS2. Therefore our vaccine specifically activates NK cells expressing KIR2DS2 and these can kill cancer cells over expressing Exportin-1. Therefore we propose that a KIR2DS2 targeting vaccine can target cancers over expressing Exportin-1.

We have also shown that using the vaccine HLA-C*0102-T2A-E3/19k-IVDVLMCHATF we can attenuate the growth of murine melanoma and lymphoma cells. We thus propose that a KIR2DS2 targeting vaccine has utility against liver cancer, melanoma and lymphoma. In general these are exemplars of tumours expressing Exportin-1, but we have not demonstrated that the NAPLVHATL is presented by these specific tumour models (unlike for the HUH7 cells). Exportin-1 has however been noted to be overexpressed in lung cancer, osteosarcoma, glioma, pancreatic cancer, ovarian cancer, cervical carcinoma, renal cell carcinoma, esophageal carcinoma, gastric carcinoma, hepatocellular carcinoma, acute myeloid/lymphoid leukemia, chronic myeloid/lymphoid leukemia, mantle cell lymphoma, plasma cell leukemia and multiple myeloma. Thus, these represent targets for this approach.

1. A method of treatment or prophylaxis of cancer, wherein the cancer overexpresses exportin-1, the method comprising the administration of:
a peptide capable of activating NK cell-mediated immunity to cancer cells that overexpress exportin-1, the peptide comprising or consisting of the amino acid sequence XⁿAX²X¹,
wherein
Xⁿ is an amino acid sequence of between 5 and 12 residues, and
X¹ is any amino acid; or leucine or phenylalanine; and
X² is alanine, threonine or serine; or
administration of one or more of a nucleic acid encoding the peptide; an immunogenic composition comprising the peptide; a complex comprising the peptide; a vesicle comprising the peptide or nucleic acid encoding the peptide; a dendritic cell comprising the peptide and/or comprising nucleic acid encoding the peptide; an activated NK cell, that has been activated by the peptide; or a virus or virus like particle comprising the peptide and/or comprising nucleic acid encoding the peptide.

2. The method of treatment according to claim 1, wherein the peptide comprises or consist of the amino acid sequence XⁿHATX^{y} wherein Xⁿ is an amino acid sequence of between 5 and 11 residues and wherein X^{y} is any amino acid, L or F.

3. The method of treatment according to claim 1, wherein the peptide comprises or consists of the sequence NAPLVHATL, IVDLMCHATF, or LNPSVAATL.

4. The method of treatment according to any preceding claim, wherein the peptide is between about 8 and about 15 amino acid residues in length.

5. The method of treatment according to any preceding claim, wherein the treatment further comprise the administration of an MHC class I molecule, or nucleic acid encoding an MHC class I molecule.

6. The method of treatment according to claim 5, wherein the MHC class I molecule is:
provided as a complex with the peptide;
encoded in the nucleic acid encoding the peptide;
provided in the immunogenic composition comprising the peptide;
provided in the vesicle comprising the peptide or encoded in the nucleic acid encoding the peptide in the vesicle;
provided in the dendritic cell comprising the peptide and/or encoded in the nucleic acid encoding the peptide in the dendritic cell; or
provided in the virus or virus like particle comprising the peptide and/or encoded in the nucleic acid encoding the peptide in the virus or virus like particle.

7. The method of treatment according to any preceding claim, wherein the activated NK cell, has been activated by the peptide complexed with an MHC class I molecule.

8. The method of treatment according to any of claims 5 to 7, wherein the MHC class I molecule comprises an MHC class I truncated at the stem region of the α3 domain.

9. The method of treatment according to any of claims 5 to 8, wherein the MHC class I molecule comprises HLA-C, or part thereof.

10. The method of treatment according to any preceding claim, wherein the cancer is a cancer selected from the group comprising liver cancer, melanoma, lymphoma, pancreatic cancer, breast cancer, lung cancer, renal cancer, leukaemia, acute myeloid/lymphoid leukaemia, chronic myeloid/lymphoid leukemia, multiple myeloma, osteosarcoma, glioma, ovarian cancer, cervical carcinoma, oesophageal carcinoma, gastric carcinoma, hepatocellular carcinoma, mantle cell lymphoma, plasma cell leukemia, and blood cancer.

11. A method of treatment or prophylaxis of a patient for cancer, wherein the cancer overexpresses exportin-1, the method comprising determining if a patient produces KIR2DS2-expressing NK cells and/or a ligand for KIR2DS2, and determining if the cancer overexpresses exportin-1;
wherein if the patient produces KIR2DS2-expressing NK cells and/or a ligand for KIR2DS2, and the cancer overexpresses exportin-1, administering:
a peptide capable of activating NK cell-mediated immunity to cancer cells that overexpress exportin-1, the peptide comprising or consisting of the amino acid sequence XⁿAX²X¹,
wherein
Xⁿ is an amino acid sequence of between 5 and 12 residues, and
X¹ is any amino acid; or leucine or phenylalanine; and
X² is alanine, threonine or serine; or
administration of one or more of a nucleic acid encoding the peptide; an immunogenic composition comprising the peptide; a complex comprising the peptide; a vesicle comprising the peptide or nucleic acid encoding the peptide; a dendritic cell comprising the peptide and/or comprising nucleic acid encoding the peptide; an activated NK cell, that has been activated by the peptide; or a virus or virus like particle comprising the peptide and/or comprising nucleic acid encoding the peptide; and
optionally wherein if the patient does not produce KIR2DS2-expressing NK cells and/or a ligand for KIR2DS2, administering to the patient activated NK cells that have been activated by the peptide.

12. A method of selecting a patient for treatment or prophylaxis with an agent arranged to activate NK cell mediated protection from cancer, wherein the cancer overexpresses exportin-1, wherein the agent is selected from:
a peptide capable of activating NK cell-mediated immunity to cancer cells that overexpress exportin-1, the peptide comprising or consisting of the amino acid sequence XⁿAX²X¹,
wherein
Xⁿ is an amino acid sequence of between 5 and 12 residues, and
X¹ is any amino acid; or leucine or phenylalanine; and
X² is alanine, threonine or serine; or
one or more of a nucleic acid encoding the peptide; an immunogenic composition comprising the peptide; a complex comprising the peptide; a vesicle comprising the peptide or nucleic acid encoding the peptide; a dendritic cell comprising the peptide and/or comprising nucleic acid encoding the peptide; an activated NK cell, that has been activated by the peptide; or a virus or virus like particle comprising the peptide and/or comprising nucleic acid encoding the peptide;
the method comprising the step of determining if the patient produces KIR2DS2-expressing NK cells and/or a ligand for KIR2DS2, and determining if the cancer overexpresses exportin-1,
wherein a patient is selected for the treatment or prophylaxis with the agent if they produce KIR2DS2-expressing NK cells and/or a ligand for KIR2DS2, and the cancer overexpresses exportin-1; and optionally wherein a patient not producing KIR2DS2-expressing NK cells and/or a ligand for KIR2DS2, and the cancer does not overexpress exportin-1, the patient is not selected for the treatment or prophylaxis with the agent, and/or is selected for an alternative cancer treatment.

13. A method of producing activated NK cells comprising exposing an NK cell expressing KIR2DS2 receptor to a peptide comprising or consisting of the sequence NAPLVHATL and optionally an MHC class I molecule.

14. A method for activating an NK cell mediated immune response of a patient for recognition of exportin-1 comprising administration of:
a peptide capable of activating NK cell-mediated immunity to cancer cells that overexpress exportin-1, the peptide comprising or consisting of the amino acid sequence XⁿAX²X¹,
wherein
Xⁿ is an amino acid sequence of between 5 and 12 residues, and
X¹ is any amino acid; or leucine or phenylalanine; and
X² is alanine, threonine or serine; or
one or more of a nucleic acid encoding the peptide; an immunogenic composition comprising the peptide; a complex comprising the peptide; a vesicle comprising the peptide or nucleic acid encoding the peptide; a dendritic cell comprising the peptide and/or comprising nucleic acid encoding the peptide; an activated NK cell, that has been activated by the peptide; or a virus or virus like particle comprising the peptide and/or comprising nucleic acid encoding the peptide;

15. Use of:
a peptide capable of activating NK cell-mediated immunity to cancer cells that overexpress exportin-1, the peptide comprising or consisting of the amino acid sequence XⁿAX²X¹,
wherein
Xⁿ is an amino acid sequence of between 5 and 12 residues, and
X¹ is any amino acid; or leucine or phenylalanine; and
X² is alanine, threonine or serine; or
one or more of a nucleic acid encoding the peptide; an immunogenic composition comprising the peptide; a complex comprising the peptide; a vesicle comprising the peptide or nucleic acid encoding the peptide; a dendritic cell comprising the peptide and/or comprising nucleic acid encoding the peptide; an activated NK cell, that has been activated by the peptide; or a virus or virus like particle comprising the peptide and/or comprising nucleic acid encoding the peptide;
for activating an NK cell mediated immune response of a patient for recognition and killing of cells overexpressing exportin-1, or a part thereof.

16. A peptide capable of activating NK cell-mediated immunity to cancer cells that overexpress exportin-1, the peptide comprising or consisting of the amino acid sequence XⁿAX²X¹,
wherein
Xⁿ is an amino acid sequence of between 5 and 12 residues, and
X¹ is any amino acid; or leucine or phenylalanine; and
X² is alanine, threonine or serine; or
one or more of a nucleic acid encoding the peptide; an immunogenic composition comprising the peptide; a complex comprising the peptide; a vesicle comprising the peptide or nucleic acid encoding the peptide; a dendritic cell comprising the peptide and/or comprising nucleic acid encoding the peptide; an activated NK cell, that has been activated by the peptide; or a virus or virus like particle comprising the peptide and/or comprising nucleic acid encoding the peptide;
for use as a vaccine against exportin-1, or cells that overexpress exportin-1, or for use to treat cancer, wherein the cancer cells overexpress exportin-1.

17. A nucleic acid encoding a peptide comprising or consisting of the amino acid sequence NAPLVHATL and an MHC class I molecule.

18. An immunogenic composition comprising a peptide comprising or consisting of the amino acid sequence NAPLVHATL and an MHC class I molecule.

19. A complex comprising a peptide comprising or consisting of the amino acid sequence NAPLVHATL and an MHC class I molecule.

20. A vesicle comprising a peptide comprising or consisting of the amino acid sequence NAPLVHATL and optionally an MHC class I molecule; and/or comprising nucleic acid encoding a peptide comprising or consisting of the amino acid sequence NAPLVHATL and optionally an MHC class I molecule.

21. A dendritic cell comprising a peptide comprising or consisting of the amino acid sequence NAPLVHATL and optionally an MHC class I molecule; and/or comprising nucleic acid encoding a peptide comprising or consisting of the amino acid sequence NAPLVHATL and optionally nucleic acid encoding the MHC class I molecule.

22. An activated NK cell, that has been activated by a peptide comprising or consisting of the amino acid sequence NAPLVHATL and optionally an MHC class I molecule.

23. A virus or virus like particle comprising a peptide comprising or consisting of the amino acid sequence NAPLVHATL and optionally an MHC class I molecule; and/or comprising nucleic acid encoding a peptide comprising or consisting of the amino acid sequence NAPLVHATL and optionally nucleic acid encoding the MHC class I molecule.


SEQUENCE LISTING (69 sequences)

SEQ ID NO: 1 - protein, 9 aa, Artificial Sequence
  Peptide
LNPSVAATL

SEQ ID NO: 2 - protein, 8 aa, Artificial Sequence
  Peptide
NPSVAATL

SEQ ID NO: 3 - protein, 7 aa, Artificial Sequence
  Peptide
PSVAATL

SEQ ID NO: 4 - protein, 9 aa, Artificial Sequence
  Peptide
VAPWNAATL

SEQ ID NO: 5 - protein, 8 aa, Artificial Sequence
  Peptide
APWNAATL

SEQ ID NO: 6 - protein, 7 aa, Artificial Sequence
  Peptide
PWNAATL

SEQ ID NO: 7 - protein, 9 aa, Artificial Sequence
  Peptide
VAPWNSATL

SEQ ID NO: 8 - protein, 8 aa, Artificial Sequence
  Peptide
APWNSATL

SEQ ID NO: 9 - protein, 7 aa, Artificial Sequence
  Peptide
PWNSATL

SEQ ID NO: 10 - protein, 9 aa, Artificial Sequence
  Peptide
VAPWNSAAL

SEQ ID NO: 11 - protein, 8 aa, Artificial Sequence
  Peptide
APWNSAAL

SEQ ID NO: 12 - protein, 7 aa, Artificial Sequence
  Peptide
PWNSAAL

SEQ ID NO: 13 - protein, 9 aa, Artificial Sequence
  Peptide
VAPWNAAAL

SEQ ID NO: 14 - protein, 8 aa, Artificial Sequence
  Peptide
APWNAAAL

SEQ ID NO: 15 - protein, 9 aa, Artificial Sequence
  Peptide
GAVPDLAWL

SEQ ID NO: 16 - protein, 9 aa, Artificial Sequence
  Peptide
GAVPDLATL

SEQ ID NO: 17 - protein, 7 aa, Artificial Sequence
  Peptide
PWNAAAL

SEQ ID NO: 18 - protein, 9 aa, Artificial Sequence
  Peptide
LNPSVAATI

SEQ ID NO: 19 - protein, 8 aa, Artificial Sequence
  Peptide
NPSVAATI

SEQ ID NO: 20 - protein, 7 aa, Artificial Sequence
  Peptide
PSVAATI

SEQ ID NO: 21 - protein, 9 aa, Artificial Sequence
  Peptide
VAPWNAATI

SEQ ID NO: 22 - protein, 8 aa, Artificial Sequence
  Peptide
APWNAATI

SEQ ID NO: 23 - protein, 7 aa, Artificial Sequence
  Peptide
PWNAATI

SEQ ID NO: 24 - protein, 9 aa, Artificial Sequence
  Peptide
VAPWNSATI

SEQ ID NO: 25 - protein, 8 aa, Artificial Sequence
  Peptide
APWNSATI

SEQ ID NO: 26 - protein, 7 aa, Artificial Sequence
  Peptide
PWNSATI

SEQ ID NO: 27 - protein, 9 aa, Artificial Sequence
  Peptide
VAPWNSAAI

SEQ ID NO: 28 - protein, 8 aa, Artificial Sequence
  Peptide
APWNSAAI

SEQ ID NO: 29 - protein, 7 aa, Artificial Sequence
  Peptide
PWNSAAI

SEQ ID NO: 30 - protein, 9 aa, Artificial Sequence
  Peptide
VAPWNSAAI

SEQ ID NO: 31 - protein, 8 aa, Artificial Sequence
  Peptide
APWNSAAI

SEQ ID NO: 32 - protein, 7 aa, Artificial Sequence
  Peptide
PWNSAAI

SEQ ID NO: 33 - protein, 9 aa, Artificial Sequence
  Peptide
LNPSVAAAL

SEQ ID NO: 34 - protein, 8 aa, Artificial Sequence
  Peptide
NPSVAAAL

SEQ ID NO: 35 - protein, 7 aa, Artificial Sequence
  Peptide
PSVAAAL

SEQ ID NO: 36 - protein, 9 aa, Artificial Sequence
  Peptide
LNPSVAASL

SEQ ID NO: 37 - protein, 8 aa, Artificial Sequence
  Peptide
NPSVAASL

SEQ ID NO: 38 - protein, 7 aa, Artificial Sequence
  Peptide
PSVAASL

SEQ ID NO: 39 - protein, 9 aa, Artificial Sequence
  Peptide
LNPSVAAWL

SEQ ID NO: 40 - protein, 8 aa, Artificial Sequence
  Peptide
NPSVAAWL

SEQ ID NO: 41 - protein, 7 aa, Artificial Sequence
  Peptide
PSVAAWL

SEQ ID NO: 42 - protein, 9 aa, Artificial Sequence
  Peptide
LNPSVAAAI

SEQ ID NO: 43 - protein, 8 aa, Artificial Sequence
  Peptide
NPSVAAAI

SEQ ID NO: 44 - protein, 7 aa, Artificial Sequence
  Peptide
PSVAAAI

SEQ ID NO: 45 - protein, 9 aa, Artificial Sequence
  Peptide
LNPSVAASI

SEQ ID NO: 46 - protein, 8 aa, Artificial Sequence
  Peptide
NPSVAASI

SEQ ID NO: 47 - protein, 7 aa, Artificial Sequence
  Peptide
PSVAASI

SEQ ID NO: 48 - protein, 9 aa, Artificial Sequence
  Peptide
LNPSVAAWI

SEQ ID NO: 49 - protein, 8 aa, Artificial Sequence
  Peptide
NPSVAAWI

SEQ ID NO: 50 - protein, 7 aa, Artificial Sequence
  Peptide
PSVAAWI

SEQ ID NO: 51 - protein, 10 aa, Artificial Sequence
  Peptide
IVDLMCHATF

SEQ ID NO: 52 - protein, 10 aa, Artificial Sequence
  Peptide
VIDAMCHATL

SEQ ID NO: 53 - protein, 9 aa, Artificial Sequence
  Peptide
AANVMAASL

SEQ ID NO: 54 - protein, 10 aa, Artificial Sequence
  Peptide
FLSDVPVATL

SEQ ID NO: 55 - protein, 9 aa, Artificial Sequence
  Peptide
  misc_feature (1)..(5): Xaa can be any naturally occurring amino acid
XXXXXHATL

SEQ ID NO: 56 - protein, 10 aa, Artificial Sequence
  Peptide
  misc_feature (1)..(6): Xaa can be any naturally occurring amino acid
XXXXXXHATL

SEQ ID NO: 57 - protein, 11 aa, Artificial Sequence
  Peptide
  misc_feature (1)..(7): Xaa can be any naturally occurring amino acid
XXXXXXXHATL

SEQ ID NO: 58 - protein, 12 aa, Artificial Sequence
  Peptide
  misc_feature (1)..(8): Xaa can be any naturally occurring amino acid
XXXXXXXXHATL

SEQ ID NO: 59 - protein, 13 aa, Artificial Sequence
  Peptide
  misc_feature (1)..(9): Xaa can be any naturally occurring amino acid
XXXXXXXXXHATL

SEQ ID NO: 60 - protein, 15 aa, Artificial Sequence
  Peptide
  misc_feature (1)..(10): Xaa can be any naturally occurring amino acid
XXXXXXXXXXNHATL

SEQ ID NO: 61 - protein, 15 aa, Artificial Sequence
  Peptide
  misc_feature (1)..(11): Xaa can be any naturally occurring amino acid
XXXXXXXXXXXHATL

SEQ ID NO: 62 - protein, 9 aa, Artificial Sequence
  Peptide
  misc_feature (1)..(5): Xaa can be any naturally occurring amino acid
XXXXXHATF

SEQ ID NO: 63 - protein, 10 aa, Artificial Sequence
  Peptide
  misc_feature (1)..(6): Xaa can be any naturally occurring amino acid
XXXXXXHATF

SEQ ID NO: 64 - protein, 11 aa, Artificial Sequence
  Peptide
  misc_feature (1)..(7): Xaa can be any naturally occurring amino acid
XXXXXXXHATF

SEQ ID NO: 65 - protein, 12 aa, Artificial Sequence
  Peptide
  misc_feature (1)..(8): Xaa can be any naturally occurring amino acid
XXXXXXXXHATF

SEQ ID NO: 66 - protein, 13 aa, Artificial Sequence
  Peptide
  misc_feature (1)..(9): Xaa can be any naturally occurring amino acid
XXXXXXXXXHATF

SEQ ID NO: 67 - protein, 14 aa, Artificial Sequence
  Peptide
  misc_feature (1)..(10): Xaa can be any naturally occurring amino acid
XXXXXXXXXXHATF

SEQ ID NO: 68 - protein, 15 aa, Artificial Sequence
  Peptide
  misc_feature (1)..(11): Xaa can be any naturally occurring amino acid
XXXXXXXXXXXHATF

SEQ ID NO: 69 - protein, 9 aa, Artificial Sequence
  Peptide
NAPLVHATL